(19) United States
(12) Patent Application Publication
(10) Pub. No.: US 2021/0302424 A1
(43) Pub. Date: Sep. 30, 2021

(54) METHODS FOR DUAL DETECTION AND DIFFERENTIATION OF INFECTION BY MYCOBACTERIUM TUBERCULOSIS COMPLEX AND NONTUBERCULOUS MYCOBACTERIA

(71) Applicant: Rutgers, The State University of New Jersey, New Brunswick, NJ (US)

(72) Inventor: Abraham Pinter, Brooklyn, NY (US)

(73) Assignee: Rutgers, The State University of New Jersey, New Brunswick, NJ (US)

(21) Appl. No.: 17/261,347

(22) PCT Filed: Jul. 18, 2019
(86) PCT No.: PCT/US19/42424

§ 371 Date: Jan. 19, 2021

Related U.S. Application Data
(60) Provisional application No. 62/700,608, filed on Jul. 19, 2018.

(51) Int. Cl. G01N 33/569; C07K 16/12

(52) U.S. Cl. CPC G01N 33/5695; C07K 2317/21; G01N 2333/35; C07K 16/1289

(57) ABSTRACT

This disclosure provides novel binary and ternary diagnostic tests with improved sensitivity and specificity for the presence of antigenic derivatives of lipo arabino mannan (LAM) present in biological fluids (e.g., sputum, serum, urine) of subjects infected with various mycobacterial pathogens, including M. tb and NTMs. The disclosed diagnostic tests detect and differentiate infection by Mycobacterium tuberculosis complex (MTBC) and nontuberculous mycobacteria (NTMs). The diagnostic tests detect different forms of LAM in the sample of patients, using capture antibodies that are either specific for TB, specific for NTMs or crossreactive with all forms of LAM, in conjunction with high-affinity species-specific or crossreactive detection antibodies.

### CROSS-REFERENCE TO RELATED APPLICATIONS

This patent document claims priority under 35 U.S.C. § 119(e) to the U.S. Provisional Patent Application No. 62/700,608, filed Jul. 19, 2018. The patent application identified above is incorporated here by reference in its entirety to provide continuity of disclosure.

### FIELD OF THE INVENTION

This disclosure relates generally to methods for detecting active infection in a subject by either Mycobacterium tuberculosis complex (MTBC) or nontuberculous mycobacteria (NTMs) and more specifically relates to methods for differentiating between these infections.

### BACKGROUND OF THE INVENTION

Tuberculosis (TB) remains one of the world's deadliest communicable diseases, with approximately one-third of the world's population currently infected with Mycobacterium tuberculosis, and an estimated 10.4 million new cases of TB disease and 1.7 million deaths from the disease in 2015. TB is also the most common cause of mortality in people living with HIV (PLHIV), with an estimated 374,000 deaths in 2016 (World Health Organization 2017). The risk of developing TB is estimated to be ˜30 times greater in PLHIV than in people without HIV. Most of the deaths from TB could have been prevented with early diagnosis. However, TB often goes undiagnosed. Globally there was an estimated 4.1 million case gap between the estimated incident and reported TB cases. This gap is due to the limitations of established tests and a lack of accurate, cheap and rapid tests suitable for the typical primary care settings in low and middle-income countries (LMICs) where TB is prevalent.

Early detection and treatment of all TB patients is a key component of the WHO's End TB Strategy. However, achieving this will require new sensitive point-of-care (POC) approaches to diagnose active TB in settings such as local health centers or clinics where patients first seek care. The absence of an accurate POC test that can quickly inform treatment decisions leads to significant patient losses at the initial stage of the care cascade. Therefore, the WHO lists a rapid biomarker-based, non-sputum-based test to detect TB as a critical unmet need in a high-priority target product profile (TPP) report (World Health Organization 2014).

There has been considerable interest in identifying mycobacterial antigens that are present in the serum or urine of patients with active TB. These antigens represent potential targets for diagnostic tests that would not require the collection of sputum samples and that could be developed in low cost immunoassay-based rapid test formats. The glycolipid lipoarabinomannan (LAM) is a major structural and antigenic component of the cell wall of members of the Mycobacterium tuberculosis-complex, and it mediates a number of important functions that promote productive infection and disease development. LAM is also an important immunodiagnostic target for detecting active infection with TB, especially in patients co-infected with HIV-1, and a potential vaccine target.

Accordingly, LAM has attracted the most attention as a diagnostic biomarker for TB. Several features of LAM make it an attractive biomarker, including: (1) it is bacterially derived, (2) abundant in the cell wall of M. tuberculosis (Mtb), (3) heat stable, and (4) has multiple epitopes, including some that are unique to M. tuberculosis. Antigenically active forms of LAM can be found in urine, blood, and sputum of many TB patients. Despite strong initial excitement following the introduction of commercial tests for detecting LAM in urine, the adoption of these tests has been limited. The primary reason for the low adoption has been the relatively poor sensitivity of the tests across the spectrum of TB cases. While some recently developed tests achieved improved sensitivity in patients with HIV, the improved sensitivity came at the expense of a decrease in specificity.

Whereas TB is well-recognized as a major pathogen that is prevalent in developing countries, the role of nontuberculous mycobacteria (NTMs) in disease is less established. NTMs are naturally-occurring organisms found in water and soil in all parts of the world, including developed countries, and can cause lung infections when a susceptible person inhales the organism from their environment. While the immune response of healthy individuals usually controls such infections, NTMs can cause disease in sensitive patient populations (e.g., immuno suppressed subjects, HIV-infected patients, transplant patients) and in subjects with compromised lung function (e.g., elderly people, cystic fibrosis patients, subjects suffering from bronchiectasis, chronic asthma and Chronic Obstructive Pulmonary Disease (COPD)). The contribution of NTMs to disease may have been underestimated, in part, due to the lack of an inexpensive, easy-to-use and sensitive assay to identify such infections.

Thus, there is a strong need for non-invasive, reliable, convenient, and inexpensive diagnostic methods and kits for detecting and differentiating infection by Mycobacterium tuberculosis complex (MTBC) and nontuberculous mycobacteria (NTMs). The inclusion of a specific test for NTMs will also result in improved sensitivity and specificity of the diagnostic test for TB, due to the ability to identify or rule out cross-reactivity with NTMs as a cause of false-positive results in the TB assay.

### SUMMARY OF THE INVENTION

This disclosure provides a method for differentiating infection by Mycobacterium tuberculosis complex (MTBC) and infection by nontuberculous mycobacteria (NTMs). The method includes: (a) obtaining, from a subject, a sample that includes a LAM-derived antigen; (b) contacting the sample, in a first test mixture, with a TB-specific capture antibody or antigen-binding portion thereof; (c) contacting the sample, in a second test mixture, with a cross-reactive capture antibody or antigen-binding portion thereof; (d) contacting the sample, in a third test mixture, with an NTM-specific capture antibody or antigen-binding portion thereof; (e) detecting binding of said LAM-derived antigen by any of these capture reagents with a detection antibody containing a specific label that provides a signal; and (f) determining whether or not the subject has an active MTBC infection, or an active NTM based on the detection of binding of a LAM-derived antigen to the cross-reactive capture antibody or antigen-binding portion thereof, and to either the TB-specific capture antibody or antigen-binding portion thereof (in case of MTBC infection), or the NTM-specific capture antibody or antigen-binding portion thereof (in case of NTM infection).

In detecting binding of step (e), the method may further include adding a first detection antibody or antigen-binding portion thereof and optionally a signal-generating reagent directed against the first detection antibody to the first test mixture for detecting the specific capture of said LAM-derived antigen by TB-specific capture antibody; adding a second detection antibody or antigen-binding portion thereof and optionally a signal-generating reagent directed against the second detection antibody to the second test mixture for detecting the specific capture of said LAM-derived antigen by the cross-reactive capture antibody to; and adding a third detection antibody or antigen-binding portion thereof and optionally a signal-generating reagent directed against the third detection antibody to the third test mixture for detecting the specific capture of said LAM-derived antigen by the NTM-specific capture antibody.

In some embodiments, the method is performed in a lateral flow assay (LFA) device. The detection reagent is labeled by conjugation to a gold particle, a latex particle, or a fluorophore, thereby resulting in a colored band that is intensified as an antigen-antibody-particle complex or an antigen-antibody-fluorophore complex accumulates at stripes on the device that contains the various capture reagents.

In some embodiments, the TB-specific capture antibody binds specifically to the LAM-derived antigen including 5-deoxy-5-methylthio-xylofuranosyl (MTX) attached to the terminal Manp by an a-(1→4) linkage. In some embodiments, the cross-reactive capture antibody binds specifically to the LAM-derived antigen including at least one of an Ara4 structure and an Ara6 structure, independent of the presence or absence of any terminal capping structures. In some embodiments, the NTM-specific capture antibody binds specifically to the LAM-derived antigen including at least one of an uncapped Ara4 structure and an Ara6 structure, but not to the corresponding capped structures.

In some embodiments, the TB-specific capture antibody can be the MoAb1 antibody. In some embodiments, the cross-reactive capture antibody is selected from the group consisting of CS-35, FIND-25, A194-01 antibodies. In some embodiments, the NTM-specific capture antibody is selected from the group consisting of 906.7, 908.1, and 922.5 antibodies that have retained their high reactivity for NTM LAM but little or no reactivity with TB LAM.

In some embodiments, the first, second, and third detection antibodies are the same antibody. In some embodiments, the first, second, and third detection antibodies are reactive with one or more LAM epitopes conserved across mycobacterial strains. In some embodiments, the first, second, or third detection antibody is the A194-01 antibody.

In some embodiments, the TB-specific capture antibody, the cross-reactive capture antibody, the NTM-specific antibody, the first detection antibody, the second detection antibody, or the third detection antibody is either an IgG, a dimeric scFv, an scFv-IgG, an IgA, an IgM antibody, or an engineered version thereof.

In some embodiments, the detecting of binding of said LAM-derived antigen to at least one of the TB-specific capture antibody or antigen-binding portion thereof, the cross-reactive capture antibody or antigen-binding portion thereof, and NTM-specific capture antibody or antigen-binding portion thereof is performed by using an method selected from the group consisting of an electrochemiluminescence assay, an enhanced chemiluminescence assay, an enzyme-linked immunosorbent assay (ELISA), and a lateral-flow assay (LFA).

In some embodiments, the sample includes an aliquot of urine or serum from the subject suspected of being actively infected by Mycobacterium tuberculosis complex (MTBC) or by nontuberculous mycobacteria (NTMs). In some embodiments, the subject is an HIV positive tuberculosis (TB) patient or an HIV positive nontuberculous mycobacteria (NTM)-infected patient.

This disclosure also provides a kit for differentiating infection by Mycobacterium tuberculosis complex (MTBC) and infection by nontuberculous mycobacteria (NTMs). The kit includes: (a) a TB-specific capture antibody or an antigen-binding fragment thereof; (b) a cross-reactive capture antibody or an antigen-binding fragment thereof; (c) an NTM-specific capture antibody or an antigen-binding fragment thereof; (d) one or more detection antibodies or antigen-binding fragments thereof that bind specifically to an unoccupied region of a LAM-derived antigen and are optionally labeled with a reporter molecule; (e) a support to which at least one of the TB-specific capture antibody, the cross-reactive capture antibody, and the NTM-specific capture antibody are bound; and (f) a buffer.

In some embodiments, the TB-specific capture antibody binds specifically to the LAM-derived antigen including 5-deoxy-5-methylthio-xylofuranosyl (MTX) attached to the terminal Manp by an a-(1→4) linkage. In some embodiments, the cross-reactive capture antibody binds specifically to the LAM-derived antigen including an Ara4 structure and/or an Ara6 structure. In some embodiments, the NTM-specific capture antibody binds specifically to the LAM-derived antigen including an Ara4 structure and/or an Ara6 structure with no terminal capping structures. In some embodiments, the one or more of the detection antibodies is the A194-01 antibody or a variant thereof.

This disclosure also provides a method for detecting infection by nontuberculous mycobacteria (NTMs). The method includes: (a) obtaining, from a subject, a sample that includes a LAM-derived antigen; (b) contacting the sample, in a test mixture, with an NTM-specific capture antibody or antigen-binding portion thereof; (c) detecting binding of said LAM-derived antigen by the NTM-specific capture antibody or antigen-binding portion thereof; and (d) determining that the subject has an active NTM based on the detection of binding of a LAM-derived antigen to the NTM-specific capture antibody or antigen-binding portion thereof and lack of binding to a TB-specific capture antibody. In detecting the binding of said LAM-derived antigen, the method may further include adding a detection antibody or antigen-binding portion thereof and optionally a signal-generating reagent directed against the detection antibody to the test mixture for detecting the specific capture of said LAM-derived antigen by an NTM-specific capture antibody.

In some embodiments, the method is in the form of a lateral flow assay. One embodiment of the lateral flow assay uses a strip that contains all three capture antibodies applied at distinct positions of the strip. When combined with a cross-reactive detection antibody, this gives two specific bands for positive samples (in addition to a control band), a common band for the cross-reactive capture reagent, and a second band that is located at different positions in the strip for the TB-specific or NTM-specific capture reagent, thus distinguishing TB from NTM infections.

### BRIEF DESCRIPTION OF THE DRAWINGS

The foregoing and other objects, features, and advantages of the present disclosure set forth herein will be apparent from the following description of particular embodiments of those inventive concepts, as illustrated in the accompanying drawings. Also, in the drawings, the like reference characters refer to the same parts throughout the different views. The drawings depict only typical embodiments of the present disclosure and, therefore, are not to be considered limiting in scope.

FIG. 1 shows a comparison of single detection assay and exemplary binary and ternary detection assays for mycobacterial LAM antigens present in the urine of patients. The assay format shown is a Lateral Flow Strip (LFA) containing either one, two, or three assay wells. The current assay uses just a single pair of antibodies that are cross-reactive for M. tuberculosis and NTM LAM, and therefore do not distinguish between TB and NTM infections. In the binary form of the assay, the first well uses a TB-specific (A) and the second well uses a cross-reactive (B) capture antibody, and this assay can detect and distinguish infections by Mycobacterium tuberculosis complex (MTBC) and by nontuberculous mycobacteria (NTMs). The ternary form of the assay also includes a third well with a capture antibody that is specific for NTM LAM (C), providing another signal for NTM samples. Since two positive signals are detected for both TB and NTM forms of infection, the ternary form of the assay provides clearer positive and negative outputs for both of these pathogens.

FIGS. 2A, 2B, 2C, and 2D (collectively “FIG. 2”) show an example of a lateral flow assay in which all three forms of capture reagents (cross-reactive, TB-specific and NTM-specific) are embedded at different positions in the strip (FIG. 2A). When the strip is developed and captured antigen visualized with the labeled detection reagent, different patterns are seen for negative samples (FIG. 2B), TB-positive samples (FIG. 2C) and NTM-positive samples (FIG. 2D).

### DETAILED DESCRIPTION OF THE INVENTION

This disclosure provides a novel binary and ternary point-of-care (POC) assay that combines probes specific only for TB or NTM LAM, and probes cross-reactive with both TB and NTM LAMs. The assay can detect different forms of LAM in the urine of patients, using capture antibodies that are either specific for TB, specific for NTMs, or cross-reactive with all forms of LAM. The assay could detect and differentiate TB infections from NTM infections. For example, a ternary assay, as described below, includes a third capture antibody specific only for NTMs. By virtue of requiring two positive signals, these tests will provide greater accuracy to the detection of TB LAM, and extend the utility of these tests to diagnosing NTMs in addition to TB, and discriminating NTM infections from infections with TB. One important aspect of this disclosure is that the binary and ternary combination of assays are not limited to the identity of any particular antibodies used but on the specificity of the antibodies for TB or NTM antigens.

### A. DEFINITIONS

The term “antibody” (Ab) as used herein is used in the broadest sense and specifically may include any immunoglobulin, whether natural or partly or wholly synthetically produced, including but not limited to monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2a, IgG2b, IgG3, IgG4, IgM, IgA1, IgA2, IgD, and IgE) and biologically relevant fragment or specific binding member thereof, including but not limited to Fab, F(ab′)₂, scFv (single chain or related entity) and (scFv)₂.

The term “antibody fragments” as used herein may include those antibody fragments obtained using techniques readily known and available to those of ordinary skill in the art, as reviewed herein. Therefore, the term “antibody” describes any polypeptide or protein comprising a portion of an intact antibody, such as the antigen binding or variable region of the intact antibody. These can be derived from natural sources, or they may be partly or wholly synthetically produced. Examples of antibody fragments include, but are not limited to, Fab, Fab′, F(ab′)₂, and Fv fragments; diabodies, and linear antibodies. In particular, as used herein, “single-chain Fv” (“sFv” or “scFv”) are antibody fragments that comprise the VII and VL antibody domains connected into a single polypeptide chain. The sFv polypeptide can further comprise, e.g., a linker such as a flexible polypeptide linker between the VII and VL domains that enables the scFv to form the desired structure for antigen binding.

The term “monoclonal antibody” or “mAb” as used herein may refer to an antibody obtained from a population of substantially homogeneous antibodies, i.e., the individual antibodies comprising the population are identical except for possible naturally occurring mutations that may be present in minor amounts.

The terms “variants,” “derivatives,” and/or “variants and/or derivatives” as used herein may refer to antibodies, antibody fragments, recombinant antibodies, whether derived from natural sources or partly or wholly synthetically produced inasmuch as the variable domains of the foregoing compounds are either structurally similar, i.e., retain a degree of homology that is at least 50%, at least 55%, at least 60%, at least 65%, at least 70%, at least 80%, at least 85%, at least 95%, at least 96%, at least 97%, at least 98%, or at least 99%, or greater sequence identity with those of the variable domains of an original unmodified antibody, and/or, independent of structural homology, may be functionally similar to the original unmodified anti-LAM antibodies, that is, they retain the ability to specifically bind to at least one epitope of LAM. For example, such variants and/or derivatives may include anti-LAM antibodies with variant Fc domains, chimeric antibodies, fusion proteins, bispecific antibodies, or other recombinant antibodies. Such variants and/or derivative antibodies may, but not necessarily, possess greater binding specificity for one or more epitope(s) of LAM, and/or may possess altered binding specificities for particular LAM antigens or epitopes than the original antibody parent.

An anti-LAM antibody may take one of numerous forms in the art, as disclosed herein. Antibodies are in part defined by the antigens to which they bind. Thus, an “anti-LAM antibody” is any such antibody which specifically binds at least one epitope of lipoarabinomannan (LAM) as described herein. It is understood in the art that an antibody is a (glycol)protein comprising at least two heavy (H) chains and two light (L) chains inter-connected by disulfide bonds, or an antigen-binding portion thereof. A heavy chain is comprised of a heavy chain variable region (VH) and a heavy chain constant region (CH1, CH2, and CH3). A light chain is comprised of a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2, and CDR3) represent hypervariable regions and are arranged from NH2 terminus to the COOH terminus as follows: FWR1, CDR1, FWR2, CDR2, FWR3, CDR3, FWR4. The variable regions of the heavy and light chains contain a binding domain that interacts with an antigen while, depending on the isotype, the constant region(s) may mediate the binding of the immunoglobulin to host tissues or factors.

It is known in the art that it is possible to manipulate monoclonal and other antibodies and use techniques of recombinant DNA technology to produce other antibodies or chimeric molecules which alter or retain the specificity of the original antibody. Such techniques may evolve introducing mutations into DNA sequences encoding the immunoglobulin variable region, or CDRs, of an antibody to the constant regions, or constant regions plus framework regions, of a different immunoglobulin.

As used herein, the term “subject” refers to any animal (e.g., a mammal), including, but not limited to humans, non-human primates, rodents, dogs, cats, horses, cows, sheep, domesticated animals and the like, which is to be the recipient of a particular treatment. Typically, the terms “subject” and “patient” are used interchangeably herein in reference to a human subject.

The term “epitope” as used herein may refer to the region of an antigen to which an antibody binds. An “antigen” refers to a substance that elicits an immunological reaction or binds to the products of that reaction.

As used herein, the terms “specific binding,” “selective binding,” “selectively binds,” and “specifically binds,” may refer to antibody binding to an epitope on a predetermined antigen but not to other antigens. Typically, the antibody (i) binds with an equilibrium dissociation constant (KD) of approximately less than 10⁻⁶ M, such as approximately less than 10⁻⁷ M, 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., a LAM epitope, as the analyte and the antibody as the ligand, or Scatchard analysis of binding of the antibody to antigen-positive cells, and (ii) binds to the predetermined antigen with an affinity that is at least two-fold greater than its affinity for binding to a non-specific antigen (e.g., BSA, casein) other than the predetermined antigen or a closely-related antigen.

### B. NONTUBERCULOUS MYCOBACTERIA (NTMS)

NTMs are prevalent all around the world and cause disease in some sensitive patient populations (e.g., immunosuppressed subjects, HIV-infected patients, transplant patients, cystic fibrosis patients, elderly people). Common human NTM pathogens that have been described include M. abscessus, M. avium complex (MAC), M. kansasii, M. fortuitum, and M. chelonae. MAC has historically been the most common NTM isolated, with M. abscessus being the second most common group isolated. In the largest U.S. survey, MAC was present in up to 72% of patients with NTM-positive sputum cultures, and in other studies the percentage of M. abscessus group reported in CF patients with NTM-positive sputum cultures ranged between 16 to 68%. Less frequently isolated species include M. kansasii and M. fortuitum. M. avium complex is more often associated with older CF patients, often diagnosed in adulthood, while the M. abscessus group is frequently seen in younger patients and those with more severe lung disease. The distribution of these strains may also be effected by geographic factors, as the M. abscessus group appears especially prevalent in Europe, and M. simiae and M. abscessus group are the most common species isolated in Israel.

Different mycobacterial species also cause disease in animals. For example, M. bovis is related to M. tb, and causes a TB-like disease in cattle. M. paratuberculosis, a sub-species of M. avium, causes Johne's disease in cattle, a chronic enteritis of cattle and sheep which clinically presents as weight loss and diarrhea that is often fatal for infected animals. M. paraTB has also been suspected as a potential cause for Crohn's disease in humans.

It is likely that the contribution of NTMs to disease has been underestimated, in part, due to the lack of an inexpensive, easy-to-use and readily available assay. This disclosure provides a tool for the initial detection of infection by these pathogens, which could also be used, for example, to follow the response of patients to chemotherapy to treat the infection. LAM levels in the urine are expected to be highest in untreated subjects with the full-blown disease. As infection clears, the level of LAM in the urine is expected to decrease and eventually disappear. For TB, the increasing outgrowth of MDR and XDR drug-resistant strains complicates drug therapy, and an effective tool for monitoring response to treatment could be very valuable in monitoring response to therapy and rapidly detecting drug resistance. For NTMs, there is also a need for the development of more effective drug therapies, and in addition to diagnosing infections, the availability of an NTM-specific point-of-care assay could facilitate the characterization of responses to existing drug and the identification of new treatments against these pathogens as well.

### C. LIPOARABINOMANNAN (LAM)

The M. tuberculosis surface glycolipid, lipoarabinomannan (LAM), is a prominent humoral target. LAM is a major structural component of the M. tuberculosis cell wall and an important mediator of functions that promote productive infection and pathogenicity. LAM contains four distinct structural domains: (1) a phosphatidylinositol anchor, (2) an a-(1→6)-linked mannan backbone of mannopyranose (Manp) residues with pendant a-(1→2)-Manp-linked side chains, (3) an arabinan chain containing multiple arabinofuranoside (Araf) residues with tetra- and hexa-Araf termini, and (4) terminal caps containing various carbohydrate motifs. Fast-growing nonpathogenic strains, such as M. smegmatis, have uncapped ends or inositol phosphate caps (PILAM), whereas virulent strains of mycobacteria, such as M. bovis and M. tuberculosis, are capped extensively with a-(1→2)-linked Manp monosaccharide, disaccharide, and trisaccharide units (ManLAM). It has been estimated that 40-70% of the nonreducing termini of LAM from pathogenic strains of M. tuberculosis are Manp capped, and analysis of the relative abundance of the different cap motifs for a virulent clinical strain showed that the dimannosyl unit was the major capping motif (˜80%), with lesser concentrations of monomannosyl and trimannosyl motifs (10-13%). M. tuberculosis ManLAM contains an additional cap modification, 5-deoxy-5-methylthio-xylofuranosyl (MTX), attached to a terminal Manp by an a-(1→4) linkage, that is present in low abundance. The MTX sugar can also be present as the corresponding oxidized derivative (MSX), in which the sulfur atom is modified to a corresponding sulfoxide.

Soluble LAM secreted from bacteria and infected cells is an important immunodiagnostic target for M. tuberculosis infection and activation. A commercial lateral flow assay that uses polyclonal antibodies raised against LAM has proven useful in diagnosing TB infection in HIV-coinfected patients with very low CD4 counts, and the use of this test allowed early detection and subsequent treatment that was associated with reduced mortality in such patients. However, the sensitivity of this assay was insufficient to detect infection in the general population. Therefore, it is not recommended by the World Health Organization as a general screening test for TB, but limited to populations with known HIV-1 infections and CD4 counts of <100. It has been proposed that the greater sensitivity of this assay in HIV+ patients may be related to TB dissemination to the kidneys, with concomitant increases in the levels of antigens present in the urine, although more recent analyses indicate that lower concentrations of LAM are also present in the urine of HIV negative TB patients. This suggests that the performance of these assays would be improved by the identification of more sensitive and specific antibodies that would increase the accuracy and allow a more widely useful POC.

The precise structure of the secreted forms of LAM are not known, but evidence indicates that they differ structurally and immunologically from the molecules purified from the bacteria. For example, although mAbs such as P30B9 or MoAb2 that are specific for dimannose-capped epitopes are sensitive capture regents for the bacterial antigen they are very inefficient at capturing the urinary LAM-derived forms of the antigen, while antibodies specific for cross-reactive epitopes, such as FIND25, can efficiently detect both bacterial an urinary forms of the antigen. This suggests that the dimannose cap-dependent epitopes are not retained in the urinary antigen. On the other hand, the MTX-dependent epitope recognized by the MoAb1 (also referred to as S4-20) antibody is efficiently retained in the urine, where it appears to be a dominant antigen. The basis for this is not clear, since the MTX sugar is directly attached to the mannose caps. The mechanistic basis of this is unknown, and possible explanations may be that the urinary antigens consists of smaller fragments of the bacterial molecule which have lost the multivalent structures required for the presentation of some epitopes, or that some of the capping elements (i.e., dimannose-capped) are degraded due to enzymatic digestion or are efficiently cleared from the circulation by binding to specific receptors, while others (i.e., MTX-modified caps) are shielded from these effects.

### D. ANTI-LAM ANTIBODIES

(a) Detection Antibodies for LAM Antigen

A194-01, isolated at Rutgers, is a highly sensitive mAb for the detection of both Mtb and NTM-derived urinary LAM-derived antigens captured by other reagents. A194-01 antibody is further described below in section E. A mAb that recognizes the same or a related epitope with higher affinity than A194-01 would provide a stronger signal and would be expected to increase the sensitivity of the detection assay. Such an antibody could have a sequence related to that of A194-01, could be a mutated version of A194-01, or could be an unrelated antibody with a similar epitope specificity and binding properties. Additional epitopes likely exist that can also be used as sensitive detection targets for captured LAM-derived antigen from both Mtb and various NTMs. Furthermore, the capture or detection reagents could consist of combinations of antibodies that recognize more restricted NTM-specific epitopes that increase the strength and breadth of detection of certain NTM species.

In general, detection antibodies for LAM antigen may include, without limitation:
(i) A mAb that binds to LAM purified from Mtb and a broad range of NTM species with high affinity;
(ii) A mAb that efficiently detects LAM-derived antigen captured from the urine of patients infected with either Mtb or a broad range of NTM species;
(iii) A mAb that binds to ManLAM and PILAM with similar or higher affinity than A194-01;
(iv) A mAb that efficiently competes for the binding of A194-01 to ManLAM and PILAM;
(v) A molecularly engineered mutant of A194-01 that detects urinary LAM-derived antigens with higher affinity than the parental A194-01 antibody;
(vi) A mAb that binds with high affinity to uncapped Ara4 and Ara6 glycoconjugates, and with moderate affinities to Ara4 and Ara6 with mono- and dimannose caps with and without MTX substitutions;
(vii) A mAb that efficiently binds to LAM-derived antigen present in urine of TB patients and captured by MoAbs or FIND25; or
(viii) A mixture of mAbs that individually detect urinary LAM-derived antigen from a limited range of mycobacterial species that when combined detect a broad range of such antigens.

(b) TB-Specific Capture Antibodies

One of the most specific characteristics of Mtb LAM is the presence of the MTX substitution on the terminal Mannose cap. It has been demonstrated that MoAb1 (aka, S4-20, originally isolated at Otsuka) is dependent on the presence of the MTX substitution and does not cross-react with LAM from either M. smegmatis or M. leprae (Choudhary A, et al. J Immunol. 2018 May 1; 200(9):3053-3066) or with LAM from a number of other NTMs (Sigal et al., J Clin Microbiol. 2018 Nov. 27; 56(12): e01338-18). In general, TB-specific capture antibodies may include, without limitation:
(i) A mAb that binds specifically to purified ManLAM but not to PILAM or LAM purified from other mycobacterial species, including M. smegmatis and M. leprae;
(ii) A mAb that binds specifically to Mtb cells and cell lysates, but not to cells and cell lysates of other mycobacterial species;
(iii) A mAb that binds specifically to synthetic glycoconjugates containing MTX attached by an a-(1→4) linkage to monomannose or dimannose-capped Ara4 or Ara6 structures, but not to the same structures missing the MTX substitution. Examples of glycoconjugates are described in Choudhary A, et al. J Immunol. 2018; or
(iv) A mAb that competes with high efficiency with the binding of MoAb1 to ManLAM or to a reactive glycoconjugate.

(c) Cross-Reactive Capture Antibodies

Cross-reactive capture antibodies may include, without limitation:
(i) A mAb that binds to a common site present in LAM purified from both ManLAM and PILAM;
(ii) A mAb that binds to a common site present in urinary LAM-derived antigen present in patients infected with both M. tb and a broad range of NTMs;
(iii) A mAb that binds with high affinity with glycoconjugates containing both uncapped Ara4 and Ara6 structures and Ara4 and Ara6 structures containing various Man and MTX-Man caps;
(iv) A mAb that efficiently captures LAM-derived antigen present in urine of patients infected with both M. tb and a broad range of NTMs; or
(v) A mAb that competes efficiently with the binding of mAbs CS-35 and FIND25 to ManLAM and PILAM.

(d) NTM-Specific Capture Antibodies

NTM-specific capture antibodies may include, without limitation:
(i) A mAb that efficiently captures LAM produced by various NTM species but not by M. tb;
(ii) A mAb that efficiently captures LAM-derived antigens present in the urine of patients infected with various NTM species but not in urine of patients infected with M. tb;
(iii) A mAb that binds with high affinity to PILAM but not to ManLAM (Examples are shown below in Table 1);
(iv) A mAb isolated from an NTM-infected patient that specifically reacts with high affinity to LAM or LAM-derived urinary antigen produced by some or all NTMs, but not by M. tb;
(v) A mixture of mAbs isolated from patients infected with different NTM species that specifically reacts with high affinity to LAM or LAM-derived urinary antigen produced by a broad range of NTMs, but not by M. tb; or
(vi) A mAb that reacts with uncapped Ara4 and Ara6 glycoconjugates, but not with similar structures modified with mannose or MTX-mannose caps

TABLE 1
Examples demonstrating the presence of monoclonal
antibodies with high affinity for a non-TB LAM
antigen with negligible reactivity with Mtb LAM

| AC 051018 Results (αFc αγμ mix) | |
|---|---|
| 10 minutes | 30 minutes |

| | ManLAM | PILAM | ManLAM | PILAM |
|---|---|---|---|---|
| P8A2 | 1.10 | 0.81 | >3.60 | >3.60 |
| P8D1 | 0.65 | 0.39 | 3.60 | 2.14 |
| P4B12 | 0.17 | 0.16 | 1.08 | 0.97 |
| P4C1 | 0.18 | 0.16 | 0.95 | 0.74 |
| P2A12 | 0.07 | 2.57 | 0.10 | >3.60 |
| P4H9 | 0.07 | 1.36 | 0.08 | >3.60 |
| P5H1 | 0.07 | 0.60 | 0.11 | 2.86 |

ManLAM purified from M. tb or PILAM purified from M. smegmatis were used to coat wells of a 96-well ELISA plate and incubated with supernatant medium from memory B cell cultures generated from cells purified from an M. tb-infected patient. The top 4 samples contained mAbs that cross-reacted with different relative specificities with both forms of LAM, while the bottom 3 samples contained mAbs that were highly reactive with PILAM but did not recognize ManLAM. This demonstrates the presence of mAbs that preferentially recognize an NTM LAM (PILAM) over M. tb LAM (ManLAM) even in cells derived in response to infection by M. tb.

### E. EXAMPLES OF ANTI-LAM ANTIBODIES

A194-01 Antibody

The A194-01 antibody is described in greater detail in patent application PCT/US2017/016058. A194-01 is a human monoclonal antibody. A194-01 antibody and its variants and/or derivatives thereof are specific for LAM. mAbs A194-01 recognized various Ara4 and Ara6 motifs. For example, A194-01 reacted strongly with the uncapped Ara4 and Ara6 structures, including the inositol phosphate-capped Ara4; and to a less degree with single Manp-capped structures, including their MTX-modified forms; and only weakly with di- and tri-Manp-capped structures. However, A194-01 does not bind strongly to structures containing only the terminal b-Araf-(1→2)-a-Araf-(1→3) structure, corresponding to the lower branch of the Ara6 structure.

A194-01 was originally isolated and purified as an IgG. The IgG isotype of A194-01 exhibited 50% maximal binding activity of the antibody at a concentration of approximately 20 ng/ml, thus signifying a high affinity for LAM. However, A194-01 may exist in a number of isotypes, as well as engineered and recombinant isotypes, including but not limited to IgG, IgA, IgM, monovalent single chain Fv (scFv) fragments, Fab proteins, divalent scFv fragments, single chain scFv fragments (monomers) wherein individual variable light and variable heavy regions are joined by e.g., a flexible linker, and dimeric scFv proteins in which two scFv monomers are joined to one another.

The IgG isotype of A194-01 recognizes a unique and complex epitope that is expressed on unmodified Ara4 and Ara6 side-chains and on side-chains bearing a single mannose. Although A194-01 does not bind well to side chains bearing di- or tri-mannose substitutions, it does react better with such structures if they are further modified with an MTX substituent. Accordingly, the IgG isotype of A194-01 binds to PILAM and ManLAM with high affinity, and also binds strongly with uncapped versions of both Ara4 and Ara6 structures, and binds less strongly to single mannose-capped and MTX-substituted Ara4/Ara6 structures, but poorly if at all to disubstituted and trisubstituted ManLAM. Without wishing to be bound by theory, the dramatically different effect of attachment of mannose versus MTX to the terminal mannose of the mono-mannosylated Ara4 structure may reflect a difference in the structure of MTX and mannose, or a difference between the a-(1→2) linkage of the mannose and a-(1→4) linkage of the MTX substitution. Engineered variants and/or derivatives of A194-01, including those that possess higher valencies, may exhibit broader epitope specificity than the A194-01 IgG isotype, and may further exhibit enhanced affinity for LAM. For example, the tetravalent scFv-IgG engineered A194-01 and the engineered IgA and IgM isotypes bind to both Ara4 and Ara6 structures with higher avidities than the A194-01 IgG isotype, and furthermore, also recognize di-mannose and tri-mannose capped structures that the IgG isotype binds to poorly.

CS-35 Antibody

Mouse mAb CS-35 antibody exhibits the broadest reactivity against the glycoconjugates. It recognizes all Ara4 and Ara6 structures with similar affinities, consistent with the broad recognition by this mAb of all three forms of LAM. CS-35 reactivity appeared to be completely insensitive to the presence or absence of capping, consistent with the previously reported specificity of CS-35 for the Ara4 (e.g., b-Araf-(1→2)-a-Araf-(1→5)-a-Araf-(1→5)-a-Araf) motif present in all reactive structures. This binding pattern is consistent with the crystal structures of CS-35 Fab in complex with Ara4 and Ara6, which showed that the nonreducing ends in Ara4 and Ara6 project away from the Ab surface, explaining why attachment of capping residues at these positions does not interfere with recognition.

FIND-25 Antibody

The FIND-25 antibody is a member of the related FIND series of antibodies. The binding of FIND-25 to LAM-derived antigen is dependent on the presence of the Ara6 motif. FIND25 had no activity against any of the Ara4 structures, but it recognized both capped and uncapped forms of Ara6. This pattern was consistent with the broad reactivity of this mAb for all three forms of LAM. Identical patterns were also obtained for three additional antibodies related to FIND25, suggesting that this group of antibodies is also clonally related. The FIND antibody family was unreactive with two additional structures in which the 1→5 arm or the 1→3 branch of the Ara6 structure was extended by insertion of five additional 1→5-linked Araf residues between the terminal b-(1→2)-linked Araf residues and the rest of the molecule. Such extended motifs have been identified in LAM from M. smegmatis. This indicates a strong dependence on the distance between the terminal b-(1→2)-linked Araf residues on either arm of the Ara6 structure and the branching point for recognition by the FIND mAbs.

MoAb1, MoAb2, and MoAb3 Antibodies

MoAb1, MoAb2, and MoAb3 are recombinant antibodies isolated by Otsuka Pharmaceutical by phage display of scFv libraries generated from rabbits (MoAb1 and MoAb3) and a chicken (MoAb2) immunized with bacillus Calmette-Guerin and panned against ManLAM. These mAbs are dependent, to various extents, on Manp capping and possessed different sensitivities to the number of Manp residues attached and the presence or absence of MTX on the terminal Manp residue. Reactivity studies with a panel of synthetic glycoconjugates showed that MoAb1 preferentially recognizes MTX-substituted dimannose- and trimannose-capped Ara4 and Ara6 structures, with lower affinities to monomannose structures containing the MTX modification, but does not recognize the corresponding structures lacking the MTX substituent. This mAb is highly specific for ManLAM, which is known to possess the MTX modification and was the only Ab that did not recognize PILAM or LepLAM. consistent with the absence of the MTX attachment in these Ags. In contrast, MoAb2 preferentially recognizes unmodified dimannose-capped Ara4 and Ara6 glyconjugate structures, with varying lower levels of sensitivity to structures capped with monomannose and trimannose motifs; these activities were greatly reduced by MTX attachment. MoAb3 bound preferentially to dimannose-capped Ara4 and Ara6 structures, with weaker reactivity to the other capped forms.

906.7, 908.1, and 922.5 Antibodies

The 900 series of mAbs (906.7, 908.1, and 922) were isolated from mice immunized with M. leprae, These mAbs recognize PILAM and LepLAM with similar efficiencies but react only weakly with M. tuberculosis ManLAM, and they possess similar glyconjugate binding patterns, suggesting that they are clonally related. These mAbs react preferentially with uncapped Ara4 and Ara6 glycoconjugate structures and with a phospho-myoinositol-capped Ara4 structure, and are less sensitive or unreactive to the Manp-capped structures. The preferential reactivities of these mAbs for the non-TB forms of LAM correlated with the absence or limited extent of Manp capping in PILAM and LepLAM, whereas Manp-capped structures are the dominant forms present in ManLAM.

### F. DIAGNOSTIC TESTS FOR DETECTING TB AND DIFFERENTIATING INFECTION BY MYCOBACTERIUM TUBERCULOSIS COMPLEX (MTBC) AND INFECTION BY NONTUBERCULOUS MYCOBACTERIA (NTMS)

Diagnostic methods with improved sensitivity and specificity for detecting TB and differentiating infection by Mycobacterium tuberculosis complex (MTBC) and infection by nontuberculous mycobacteria (NTMs) are disclosed. The method includes obtaining from a subject a sample that comprises a LAM-derived antigen. The LAM-derived antigen may be derived from any mycobacterial species, including M. tuberculosis, M. smegmatis, M. avium, and M. abscessus. The LAM-derived antigen may comprise a LAM epitope that is related to PILAM, ManLAM, or uncapped/unmodified LAM, or a portion thereof. In some embodiments, the LAM-derived antigen may include Ara4 and/or Ara6 structures. In some embodiments, the LAM epitope is one or more uncapped arabinose chains. In some embodiments, the LAM epitope comprises at least one methylthioxylose (MTX) or a related methylsulfonyl-xylofuranosyl (MSX) substitution. In some embodiments, the LAM epitope comprises at least one phosphoinositol substitution. In some embodiments, the LAM epitope comprises at least one phosphatidyl-myoinositol substitution (PILAM). In some embodiments, the LAM epitope is an arabinose chain capped with at least one mannose, i.e., mannosylated Man-LAM epitope. In further embodiments, the capped arabinose chain comprises Ara4 and/or Ara6 structures. In some embodiments, the Man-LAM epitope comprises mono-mannose substituted side chains, di-mannose substituted side chains, tri-mannose substituted side chains or combinations thereof. In some embodiments, the Man-LAM epitope comprises di-mannose or tri-mannose capped Ara4 and/or Ara6 structures. In some embodiments, the Man-LAM epitope is di-mannose capped Ara6.

The sample can be a biological sample. Biological sample refers to a sample obtained from an organism (e.g., patient) or from components (e.g., cells) of an organism. The sample may be of any biological tissue, cell(s) or fluid. The sample may be a “clinical sample” which is a sample derived from a subject, such as a human patient. Such samples include, but are not limited to, saliva, sputum, blood, blood cells (e.g., white cells), amniotic fluid, plasma, semen, bone marrow, tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.” A biological sample may also include a substantially purified or isolated protein, membrane preparation, or cell culture.

In some embodiments, the sample comprises an aliquot of urine or serum from a patient suspected of being actively infected by Mycobacterium tuberculosis complex (MTBC) or by nontuberculous mycobacteria (NTMs). In some embodiments, the subject is an HIV positive tuberculosis (TB) patient or an HIV positive nontuberculous mycobacteria (NTMs)-infected patient.

Binary Assay

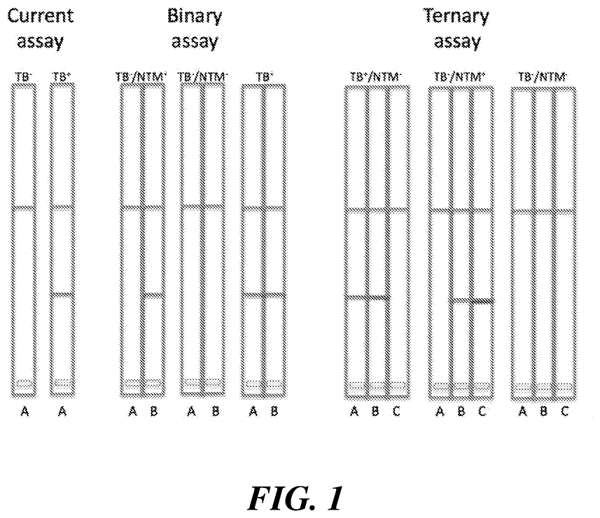

As shown in FIG. 1 (middle panel), the binary assay includes: (a) obtaining, from a subject, a sample that may or may not contain a LAM-derived antigen; (b) applying that sample to a strip (lane A) that contains a TB-specific capture antibody or antibody fragment thereof, (c) applying that sample to a second strip (lane B) that contains a cross-reactive antibody or antibody fragment thereof; (d) applying a specific detection antibody or antibody fragment thereof that recognizes a separate portion on the captured antigen than the capture antibody; and (e) direct visualization of the detection antibody by its associated label or by further amplification of that label; and (f) determining that the subject has the infection by MTBC or the infection by NTMs based on the presence of a specific signal in both lanes A and B (signifying MTBC+) or in lane B only (signifying MTBC−, NTM+). The absence of such a signal in either lane A or lane B would signify no infection by either MTBC or by NTM.

In some embodiments, the sample comprises an aliquot of urine or serum from the subject suspected of being actively infected by Mycobacterium tuberculosis complex (MTBC) or by nontuberculous mycobacteria (NTMs). In some embodiments, the subject is an HIV positive tuberculosis (TB) patient or an HIV positive nontuberculous mycobacteria (NTMs)-infected patient.

Virulent strains of the Mycobacterium tuberculosis-complex are extensively capped with mono-, di-, and tri-α(1→2)-D-Manp saccharide units, while fast-growing non-pathogenic strains like M. smegmatis have uncapped ends or phosphatidyl-myoinositol caps (PILAM). It has been estimated that 40-70% of the nonreducing termini of LAM from pathogenic strains of the Mycobacterium tuberculosis-complex are mannose-capped, and analysis of the relative abundance of the different cap motifs for the virulent MT103 clinical strain showed that the dimannosyl unit was the major structural motif (75-80%), while the mannosyl and the trimannosyl motifs were present at lower concentrations (10-13%). This extensive mannose capping differentiates virulent strains of the Mycobacterium tuberculosis-complex from non-virulent/non-pathogenic strains, such as M. smegmatis. LAM found in the strain M. tb also contain in low abundance a unique capping structure linked to some terminal mannose residues, 5-deoxy-5-methyl-thio-pentofuranose (MTX), which may present a unique marker to identify M. tuberculosis.

In some embodiments, the TB-specific capture antibody binds specifically to the LAM-derived antigen comprising a terminal Manp cap. In some embodiments, the TB-specific capture antibody binds specifically to the LAM-derived antigen comprising 5-deoxy-5-methylthio-xylofuranosyl (MTX) attached to the terminal Manp by an a-(1→4) linkage.

In contrast, the cross-reactive LAM-derived antigen may include one or more epitopes conserved across mycobacterial strains. In some embodiments, the cross-reactive LAM-derived antigen comprises Ara4 and/or Ara6 structures, both with and without a terminal Manp cap, which is or is not further modified by a 5-deoxy-5-methylthio-xylofuranosyl (MTX) sugar attached to the terminal Manp by an a-(1→4) linkage. In some embodiments of the present disclosed binary assay, the cross-reactive capture antibody binds specifically to the LAM-derived antigen comprising at least one of an Ara4 structure and an Ara6 structure.

One of ordinary skill in the art will understand that these binary assays are not limited to the identity/use of particular antibodies. In some embodiments, the TB-specific capture antibody is the MoAb1 antibody. In some embodiments, the cross-reactive capture antibody is selected from the group consisting of CS-35, FIND-25, A194-01 antibodies.

In some embodiments, the first and second detection antibodies are the same antibody. In some embodiments, the first and second detection antibodies are reactive with one or more LAM epitopes conserved across mycobacterial strains. In some embodiments, the first or second detection antibody is the A194-01 antibody.

In some embodiments, the TB-specific capture antibody, the cross-reactive capture antibody, the first detection antibody, or the second detection antibody is an IgG, a dimeric scFv, a scFv-IgG, an IgA, or an IgM antibody, or an engineered version thereof.

As understood by a person having ordinary skill in the art, any suitable assay configurations can be used to implement the disclosed methods for differentiating infection by MTBCs and infection by NTMs. In some embodiments, the method can be carried out on an apparatus including a conventional lateral flow test strip, e.g., an immunoassay test strip, as an assay medium. In some embodiments, the method can be carried out on a multi-well plate (e.g., a 3-, 6-, 8-, 24-, 96-, 384- or 1536-well plate), and the wells of the plate can further comprise a plurality of distinct assay domains to accommodate measurements of multiple samples from a single subject or from different subjects.

In some embodiments, detecting binding of at least one of the TB-specific capture antibody or antigen-binding portion thereof and the cross-reactive capture antibody or antigen-binding portion thereof to said LAM-derived antigen is performed by using an assay to detect a formation of antigen/antibody complexes. In some embodiments, the assay is selected from the group consisting of an electrochemiluminescence assay, an enhanced chemiluminescence assay, an enzyme-linked immunosorbent assay (ELISA), and a lateral-flow assay (LFA).

Electrochemiluminescence is produced by electrochemical reactions in solutions. In electrogenerated chemiluminescence, electrochemically generated intermediates undergo a highly exergonic reaction to produce an electronically excited state that then emits light upon relaxation to a lower-level state. This wavelength of the emitted photon of light corresponds to the energy gap between these two states. Electrogenerated chemiluminescence excitation can be caused by energetic electron transfer reactions of electrogenerated species. Such luminescence excitation is a form of chemiluminescence where one/all reactants are produced electrochemically on the electrodes. Electrogenerated chemiluminescence is usually observed during application of a potential to electrodes of an electrochemical cell that contains a solution of luminescent species in an aprotic organic solvent. Inorganic solvents, both oxidized and reduced forms of luminescent species can be produced at different electrodes simultaneously or at a single one by sweeping its potential between oxidation and reduction. The excitation energy is obtained from recombination of oxidized and reduced species.

Enhanced chemiluminescence is a common technique for a variety of detection assays in biology. A horseradish peroxidase enzyme (HRP) is tethered to an antibody that specifically recognizes the molecule of interest, and the complex is added to wells containing immobilized or captured antigen. After washing away unbound complex, the captured enzyme complex then catalyzes the conversion of the enhanced chemiluminescent substrate into a sensitized reagent in the vicinity of the molecule of interest, which on further oxidation by hydrogen peroxide, produces a triplet (excited) carbonyl, which emits light when it decays to the singlet carbonyl. Enhanced chemiluminescence allows detection of minute quantities of a biomolecule. Proteins can be detected down to femtomole quantities, well below the detection limit for most assay systems.

As used herein, the term “ELISA” (enzyme-linked immunosorbent assay) involves detection of an “analyte” (i.e. the specific substance whose presence is being quantitatively or qualitatively analyzed) in a liquid sample by a method that continues to use liquid reagents during the “analysis” (i.e. controlled sequence of biochemical reactions that will generate a signal which can be easily quantified and interpreted as a measure of the amount of analyte in the sample) inside a reaction chamber or well needed to keep the reactants contained. Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are unspecifically bound. After the final wash step, the plate is developed by adding an enzymatic substrate to produce a visible signal, which indicates the quantity of antigen in the sample.

Lateral flow assay (LFA) also known as lateral flow immunochromatographic assays, are simple devices intended to detect the presence (or absence) of a target analyte in the sample (matrix) without the need for specialized and costly equipment, though many lab-based applications exist that are supported by reading equipment. LFA can be used for medical diagnostics either for home testing, point of care testing, or laboratory use. The analyte is applied to a series of capillary beds, such as pieces of porous paper, microstructured polymer, or sintered polymer, which can transport fluid (e.g., urine) spontaneously. LFA can operate as either competitive or sandwich assays. Routinely, the target antigen is allowed to contact the detection reagent, which can consist of the detection antibody modified with a label, such as a gold particle, which allows it to be detected visually. The antibody-antigen complexes migrate up the strip, until they are captured and concentrated by the immobilized capture antibody, forming a visible band. The gold label can be further amplified by formation of a larger silver complex to give an enhanced signal (such as in the Fujifilm assay).

Ternary Assay

As shown in FIG. 1 (right panel), the ternary assay includes: (a) obtaining, from a subject, a sample that comprises a LAM-derived antigen; (b) contacting the sample, in a first test mixture (lane A), with a TB-specific capture antibody or antigen-binding portion thereof; (c) contacting the sample, in a second test mixture (lane B), with a cross-reactive capture antibody or antigen-binding portion thereof; (d) contacting the sample, in a third test mixture (lane C), with an NTM-specific capture antibody or antigen-binding portion thereof; (e) detecting binding of said LAM-derived antigen by the TB-specific capture antibody or antigen-binding portion thereof, by the cross-reactive capture antibody or antigen-binding portion thereof, or by the NTM-specific capture antibody or antigen-binding portion thereof; and (f) determining that the subject has an active MTBC infection, or an active NTM based on the detection of binding of a LAM-derived antigen to the cross-reactive capture antibody or antigen-binding portion thereof, and to either the TB-specific capture antibody or antigen-binding portion thereof (in case of MTBC infection), or the NTM-specific capture antibody or antigen-binding portion thereof (in case of NTM infection).

In detecting binding of step (e), the method may further include adding a first detection antibody or antigen-binding portion thereof and optionally a signal-generating reagent directed against the first detection antibody to the first test mixture for detecting the specific capture of said LAM-derived antigen by TB-specific capture antibody; adding a second detection antibody or antigen-binding portion thereof and optionally a signal-generating reagent directed against the second detection antibody to the second test mixture for detecting the specific capture of said LAM-derived antigen by the cross-reactive capture antibody to; and adding a third detection antibody or antigen-binding portion thereof and optionally a signal-generating reagent directed against the third detection antibody to the third test mixture for detecting the specific capture of said LAM-derived antigen by the NTM-specific capture antibody.

In some embodiments, the sample comprises an aliquot of urine or serum from the subject suspected of being actively infected by Mycobacterium tuberculosis complex (MTBC) or by nontuberculous mycobacteria (NTMs). In some embodiments, the subject is an HIV positive tuberculosis (TB) patient or an HIV positive nontuberculous mycobacteria (NTMs)-infected patient.

In some embodiments, the sample is treated in ways to allow liberation of the antigen from pre-formed complexes, to enhance its interactions with the capture and detection antibodies. This treatment can consist of heat treatment, protease treatments, or treatment with high or low pH buffers or chaotropic salts, to denature protein and degrade protein components complexed with the antigen, and dissociate protein or lipid complexes that sequester the antigen.

In some embodiments, the sample is preconcentrated, to enhance recognition by the capture and detection reagents of low concentrations of the antigen. This preconcentration can be performed by passing the sample over a bed containing a monoclonal antibody, such as A194-01, that captures and concentrated the antigen, or by using chemically baited hydrogel nanocages which have a high affinity for the antigen. The captured antigen can be released by treatment with reagents such as low pH that dissociate the interaction between the antigen and the capture reagent, and after neutralization of the pH, applied to the standard assay medium for more sensitive detection.

In some embodiments, the TB-specific capture antibody binds specifically to the LAM-derived antigen comprising a terminal Manp cap. In some embodiments, the TB-specific capture antibody binds specifically to the LAM-derived antigen comprising 5-deoxy-5-methylthio-xylofuranosyl (MTX) attached to the terminal Manp by an a-(1→4) linkage. In some embodiments, the cross-reactive capture antibody binds specifically to the LAM-derived antigen comprising at least one of an Ara4 structure and an Ara6 structure, independent of the presence or absence of any terminal capping structures. In some embodiments, the NTM-specific capture antibody binds specifically to the LAM-derived antigen comprising at least one of an uncapped Ara4 structure and an Ara6 structure, but not to the corresponding capped structures.

One of ordinary skill in the art will understand that these binary assays are not limited to the identity/use of particular antibodies. In some embodiments, the TB-specific capture antibody is the MoAb1 antibody. In some embodiments, the cross-reactive capture antibody is selected from the group consisting of CS-35, FIND-25, A194-01 antibodies. In some embodiments, the NTM-specific capture antibody is selected from the group consisting of 906.7, 908.1, and 922.5 antibodies.

In some embodiments, the first, second, and third detection antibodies are the same antibody. In some embodiments, the first, second, and third detection antibodies are reactive with one or more LAM epitopes conserved across mycobacterial strains. In some embodiments, the first, second, or third detection antibody is A194-01 antibody.

In some embodiments, the TB-specific capture antibody, the cross-reactive capture antibody, the NTM-specific antibody, the first detection antibody, the second detection antibody, or the third detection antibody is an IgG, a dimeric scFv, a scFv-IgG, an IgA, or an IgM antibody, or an engineered version thereof.

As understood by a person having ordinary skill in the art, any suitable assay configurations can be used to implement the disclosed methods for differentiating infection by MTBCs and infection by NTMs. In some embodiments, the method can be carried out on an apparatus including a conventional lateral flow test strip, e.g., an immunoassay test strip, as an assay medium. In some embodiments, the method can be carried out on a multi-well plate (e.g., a 3-, 6-, 8-, 24-, 96-, 384- or 1536-well plate), and the wells of the plate can further comprise a plurality of distinct assay domains to accommodate measurements of multiple samples from a single subject or from different subjects.

In some embodiments, detecting binding of at least one of the TB-specific capture antibody or antigen-binding portion thereof, the cross-reactive capture antibody or antigen-binding portion thereof, and NTM-specific capture antibody or antigen-binding portion thereof to said LAM-derived antigen is performed by using an assay to detect a formation of antigen/antibody complexes. In some embodiments, the assay is selected from the group consisting of an electrochemiluminescence assay, an enhanced chemiluminescence assay, an enzyme-linked immunosorbent assay (ELISA), and a lateral-flow assay (LFA).

FIG. 1 shows a comparison of single detection assay and exemplary binary and ternary detection assays for mycobacterial LAM antigen present in the urine of patients. The assay format shown is a Lateral Flow Strip (LFA) containing either one, two, or three assay wells. The ternary form of the assay uses a combination of a TB-specific (A), a cross-reactive (B), and an NTM-specific capture antibody (C), and can detect and distinguish infections by Mycobacterium tuberculosis complex (MTBC) and by nontuberculous mycobacteria (NTMs). The ternary form of the assay provides clearer positive and negative outputs for both TB and NTMs since two positive signals are detected for both forms of infection, and two negative signals are obtained for an uninfected sample. By way of example, the antibody pair including a TB-specific anti-LAM antibody can be the MoAb1/A194-01 antibody pair, the antibody pair including a cross-reactive anti-LAM antibody can be the FIND-25/A194-01 antibody pair, and the antibody pair including an NTM-specific anti-LAM antibody can be the 906.7/A194-01 antibody pair.

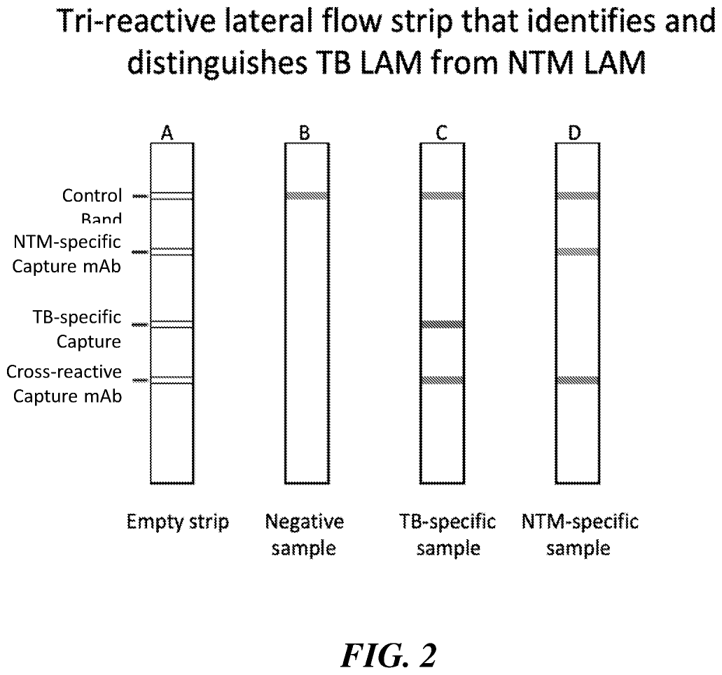

FIG. 2 shows an example of the lateral flow assay, where all three capture antibodies are placed at different positions of a single strip, and a single sample is applied and captured by reactive antibodies. A cross-reactive detection reagent, such as A194-01, is included and labels the captured antigens. The position of the band on the strip indicates which of the capture antibodies are associated with antigen. Both M. tb-derived LAM and NTM-derived LAM should give 2 bands, one at the position of the cross-reactive capture reagent, and a second at the position of either the TB-specific or NTM-specific capture reagent. The presence of both the cross-reactive band and the position of the second band thus determines whether the antigen is derived from M. tb or an NTM.

### G. EXAMPLES

LAM Ags (ManLAM, PILAM, and LepLAM) and BSA glycoconjugates were diluted in 0.05 M carbonate-bicarbonate (pH 9.6) buffer and plated at a concentration of 50 ng per well in 96-well MICROLON 200 clear round-bottom ELISA plates (catalog number 650001; Greiner Bio-One). After overnight incubation of plates at 4° C., wells were washed with PBS (pH 7.4) containing 0.05% Tween-20 (PBST) and blocked with solutions of 2% nonfat dry milk powder (Carnation) or 2% BSA (product number A3059; Sigma) in PBS buffer. Assays blocked with 2% nonfat dry milk powder had lower backgrounds but were less sensitive than those performed with BSA; overall, similar patterns were obtained. PBST-washed plates were incubated for 1 h at 37° C. with appropriate dilutions of mAbs, washed with PBST, and incubated for 30 min with a 1:1000 dilution of alkaline phosphatase (AP)-conjugated goat anti-human IgG (Fcg), IgM (Fcm), or IgA (Fca) chain-specific secondary Abs (Jackson ImmunoResearch). The plates were developed with p-nitrophenyl phosphate substrate in diethanolamine buffer (pH 9.8), and OD was measured at 405 nm on a UV-VIS spectrophotometer (Tecan SLT). Titers were defined, after subtracting the background OD₄₀₅ of the BSA-coated plate, as the reciprocal dilution that produced an OD₄₀₅ 50% of the plateau signal and were determined by exponential interpolation. Glycan- and Ab-blocking experiments were performed as described above using 2% BSA as blocking agent. For Ab-competition and LAM-capture assays, biotinylated mAbs were used as probes and detected with AP-conjugated streptavidin.

LAM-capture assays are performed using a sensitive electro-chemiluminescence detection method to enhance the signals. Assays are run in white LumiNunc 96-Well Plates (catalog number 437796; Thermo Fisher) with 2% blotto (carnation non-fat dry milk) used as blocking agent. These assays use various capture Abs plated out at 10 ug/ml and biotinylated A194-01 F(ab′)2 (0.5 mg/ml) as the detection reagent. HRP-streptavidin (Jackson ImmunoResearch Labs) is used at a 1:2,400 dilution. Plates are developed with ELISABright Substrate (catalog number K-16025-025; Advansta) and read immediately. Relative light units (RLU) are measured using a HARTA MicroLumi L2 Luminometer set for 100 msec/well at low gain).

Human urine samples were obtained from a panel of HIV-1⁺TB⁺ patients, with at least one smear test positive for acid-fast bacilli, identified in pulmonary clinics at the Texas-Mexico border and from a healthy volunteer. All but one of the patients were on anti-TB drugs for periods of time ranging from several days to 5 weeks. Human mAbs were isolated from patients with active TB disease enrolled at the Lattimore Clinic at the Global Tuberculosis Institute. Active TB disease was defined by culture-proven TB disease or a diagnosis of clinical TB. Informed written consent was obtained from all participants, and the study was approved by the Rutgers University, University of Texas Health Science Center at Houston, and Secretaria de Salud de Tamaulipas Institutional Review Boards.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used in this document, the singular forms “a,” “an,” and “the” include plural references unless the context clearly dictates otherwise. Unless defined otherwise, all technical and scientific terms used herein have the same meanings as commonly understood by one of ordinary skill in the art.

All of the methods and apparatus disclosed and claimed herein can be made and executed without undue experimentation in light of the present disclosure. While the invention has been described in terms of preferred embodiments, it will be apparent to those having ordinary skill in the art that variations may be applied to the apparatus, methods, and sequence of steps of the method without departing from the concept, spirit, and scope of the invention. More specifically, it will be apparent that certain components may be added to, combined with, or substituted for the components described herein while the same or similar results would be achieved. All such similar substitutes and modifications apparent to those having ordinary skill in the art are deemed to be within the spirit, scope, and concept of the invention as defined.

The features and functions disclosed above, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

What is claimed is:

1. A method for differentiating infection by Mycobacterium tuberculosis complex (MTBC) and infection by nontuberculous mycobacteria (NTMs), comprising:
(a) obtaining, from a subject, a sample that comprises a LAM-derived antigen;
(b) contacting the sample, in a first test mixture, with a TB-specific capture antibody or antigen-binding portion thereof;
(c) contacting the sample, in a second test mixture, with a cross-reactive capture antibody or antigen-binding portion thereof;
(d) contacting the sample, in a third test mixture, with an NTM-specific capture antibody or antigen-binding portion thereof;
(e) detecting binding of said LAM-derived antigen by the TB-specific capture antibody or antigen-binding portion thereof, by the cross-reactive capture antibody or antigen-binding portion thereof, or by the NTM-specific capture antibody or antigen-binding portion thereof;
(f) determining that the subject has an active MTBC infection, or an active NTM based on the detection of binding of a LAM-derived antigen to the cross-reactive capture antibody or antigen-binding portion thereof, and to either the TB-specific capture antibody or antigen-binding portion thereof (in case of MTBC infection) or the NTM-specific capture antibody or antigen binding portion thereof (in case of NTM infection).

2. The method of claim 1, wherein detecting binding of step (e) further comprises:
adding a first detection antibody or antigen-binding portion thereof and optionally a signal generating reagent directed against the first detection antibody to the first test mixture for detecting the specific capture of said LAM-derived antigen by TB-specific capture antibody;
adding a second detection antibody or antigen-binding portion thereof and optionally a signal-generating reagent directed against the second detection antibody to the second test mixture for detecting the specific capture of said LAM-derived antigen by the cross-reactive capture antibody to; and
adding a third detection antibody or antigen-binding portion thereof and optionally a signal-generating reagent directed against the third detection antibody to the third test mixture for detecting the specific capture of said LAM-derived antigen by the NTM-specific capture antibody.

3. The method of claim 1, wherein the method is performed in a lateral flow assay (LFA) device and the detection reagent is labeled by conjugation to a gold particle, a latex particle, or a fluorophore, thereby a colored band is intensified as an antigen-antibody-particle complex or an antigen-antibody-fluorophore complex accumulates at stripes containing the various capture reagents.

4. The method of claim 1, wherein the TB-specific capture antibody binds specifically to the LAM-derived antigen comprising 5-deoxy-5-methylthio-xylofuranosyl (MTX) attached to the terminal Manp by an a-(1→4) linkage.

5. The method of claim 1, wherein the cross-reactive capture antibody binds specifically to the LAM-derived antigen comprising at least one of an Ara4 structure and an Ara6 structure, independent of the presence or absence of any terminal capping structures.

6. The method of claim 1, wherein the NTM-specific capture antibody binds specifically to the LAM-derived antigen comprising at least one of an uncapped Ara4 structure and an Ara6 structure, but not to the corresponding capped structures.

7. The method of claim 1, wherein the TB-specific capture antibody is MoAb1.

8. The method of claim 1, wherein the cross-reactive capture antibody is selected from the group consisting of CS-35, FIND-25, and A194-01 antibodies.

9. The method of claim 1, wherein the NTM-specific capture antibody is selected from the group consisting of 906.7, 908.1, and 922.5 antibodies.

10. The method of claim 2, wherein the first, second, and third detection antibodies are the same antibody.

11. The method of claim 2, wherein the first, second, and third detection antibodies are reactive with one or more LAM epitopes conserved across mycobacterial strains.

12. The method of claim 2, wherein the first, second, or third detection antibody is the A194-01 antibody.

13. The method of claim 3, wherein the detection reagent is a mixture of antibodies specific for individual or groups of LAM antigens from various TB and NTM species, that in aggregate possesses universal or very broad reactivity for all forms of the antigen.

14. The method of claim 1, wherein detecting binding of at least one of the TB-specific capture antibody or antigen-binding portion thereof, the cross-reactive capture antibody or antigen-binding portion thereof, and NTM-specific capture antibody or antigen binding portion thereof to said LAM-derived antigen is performed by using an assay to detect a formation of antigen/antibody complexes.

15. The method of claim 14, wherein the assay is selected from the group consisting of an electrochemiluminescence assay, an enhanced chemiluminescence assay, an enzyme-linked immunosorbent assay (ELISA), and a lateral-flow assay (LFA).

16. The method of claim 1, wherein the sample comprises an aliquot of urine or serum from the subject suspected of being actively infected by Mycobacterium tuberculosis complex (MTBC) or by nontuberculous mycobacteria (NTM).

17. The method of claim 1, wherein the subject is an HIV positive tuberculosis (TB) patient or an HIV positive nontuberculous mycobacteria (NTM)-infected patient.

18. The method of claim 2, wherein the TB-specific capture antibody, the cross-reactive capture antibody, the NTM-specific antibody, the first detection antibody, the second detection antibody, or the third detection antibody is an IgG, a dimeric scFv, a scFv-IgG, an IgA, or an IgM antibody, or an engineered version thereof.

19. A kit for differentiating infection by Mycobacterium tuberculosis complex (MTBC) and infection by nontuberculous mycobacteria (NTMs), comprising:
(a) a TB-specific capture antibody or an antigen-binding fragment thereof;
(b) a cross-reactive capture antibody or an antigen-binding fragment thereof;
(c) an NTM-specific capture antibody or an antigen-binding fragment thereof;
(d) one or more detection antibodies or antigen-binding fragments thereof that bind specifically to an unoccupied region of a LAM-derived antigen and are optionally labeled with a reporter molecule;
(e) a support to which at least one of the TB-specific capture antibody, the cross reactive capture antibody, and the NTM-specific capture antibody are bound; and
(f) a buffer.

20. The kit of claim 19, wherein the TB-specific capture antibody binds specifically to the LAM-derived antigen comprising 5-deoxy-5-methylthio-xylofuranosyl (MTX) attached to the terminal Manp by an a-(1→4) linkage.

21. The kit of claim 19, wherein the cross-reactive capture antibody binds specifically to the LAM-derived antigen comprising at least one of an Ara4 structure and an Ara6 structure.

22. The kit of claim 19, wherein the NTM-specific capture antibody binds specifically to the LAM-derived antigen comprising at least one of an Ara4 structure and an Ara6 structure with no terminal capping structures.

23. The kit of claim 19, wherein the one or more detection antibodies are A194-01 antibody.

24. A method for detecting infection by nontuberculous mycobacteria (NTMs), comprising:
(a) obtaining, from a subject, a sample that comprises a LAM-derived antigen;
(b) contacting the sample, in a test mixture, with an NTM-specific capture antibody or antigen-binding portion thereof;
(c) detecting binding of said LAM-derived antigen by the NTM-specific capture antibody or antigen-binding portion thereof; and
(d) determining that the subject has an active NTM based on the detection of binding of a LAM-derived antigen to the NTM-specific capture antibody or antigen-binding portion thereof.

25. The method of claim 24, wherein detecting binding of step (c) further comprises:
adding a detection antibody or antigen-binding portion thereof and optionally a signal-generating reagent directed against the detection antibody to the test mixture for detecting the specific capture of said LAM-derived antigen by an NTM-specific capture antibody.
